(19) United States
(12) Patent Application Publication
(10) Pub. No.: US 2011/0077470 A1
(43) Pub. Date: Mar. 31, 2011

(54) Patient Monitor Symmetry Control

(75) Inventors: Tashriq Hussain, Parker, CO (US); Clark R. Baker, JR., Newman, CA (US)

(73) Assignee: Nellcor Puritan Bennett LLC, Boulder, CO (US)

(21) Appl. No.: 12/570,410

(22) Filed: Sep. 30, 2009

(51) Int. Cl. A61B 5/00

(52) U.S. Cl. 600/300

(57) ABSTRACT

Embodiments of the present disclosure relate to customization of user interfaces for medical devices. According to certain embodiments, patient monitors may include a symmetry control feature that adjusts the position of touch sensitive inputs shown on a display of the patient monitor. The symmetry control feature may allow a user to move the touch sensitive inputs from one side of the display to another to allow a user to access the touch sensitive inputs without blocking a portion of the display that displays patient physiological data. Further, in certain embodiments, the symmetry control feature may vary the location of one or more touch sensitive inputs based on user identification information received through a communication interface.

### BACKGROUND

The present disclosure relates generally to patient monitors, and, more particularly, to customizable patient monitor user interfaces.

This section is intended to introduce the reader to aspects of the art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

In the field of medicine, doctors often desire to monitor certain physiological characteristics of their patients. Accordingly, a wide variety of devices have been developed for monitoring many such characteristics of a patient. Such devices provide doctors and other healthcare personnel with the information they need to provide the best possible healthcare for their patients. As a result, such monitoring devices have become an indispensable part of modern medicine.

Patient monitors include medical devices that facilitate measurement and observation of patient physiological data. For example, pulse oximeters are a type of patient monitor. A typical patient monitor cooperates with a sensor to detect and display a patient's vital signs (e.g., temperature, pulse rate, respiratory rate) and/or other physiological measurements (e.g., water content of tissue, blood oxygen level) for observation by a user (e.g., clinician). For example, pulse oximeters are generally utilized with related sensors to detect and monitor a patient's functional oxygen saturation of arterial hemoglobin (i.e., SpO2) and pulse rate. Other types of patient monitors, such as blood pressure monitors, may be utilized to detect and monitor other physiological parameters. Further, the patient monitors may be incorporated into other types of medical devices, such as mechanical ventilators and anesthesia machines, among others.

A patient monitor may include a screen that displays information relating to operation and use of the patient monitor. A typical patient monitor screen may display operational data that is instructive and that facilitates operation of the monitor by a user. For example, the operational data may include status indicators and instructional data relating to the monitor itself and/or monitor applications (e.g., a power indicator, an alarm silenced icon, and a battery low indicator). The screen may also display measurement data from a patient being monitored. For example, the measurement data may include information relating to a physiological feature of the patient being monitored. Specifically, the screen may display a graph or trend (e.g., a pulse rate trend, and/or a plethysmographic waveform) of data relating to particular measured physiological parameters.

A patient caretaker may navigate through various screens of a patient monitor using input devices, such as buttons, to view operational data and/or to change operating parameters, such as alarm limits, of the patient monitor. However, due to the location of the input devices, a caretaker may reach across the screen to access the input devices, which may obstruct portions of the screen.

### BRIEF DESCRIPTION OF THE DRAWINGS

Advantages of the disclosure may become apparent upon reading the following detailed description and upon reference to the drawings in which:

FIG. 1 is a perspective view of an embodiment of a patient monitor that may employ a symmetry control feature;

FIG. 2 is a perspective view of the patient monitor of FIG. 1 displaying a screen after activation of the symmetry control feature;

FIG. 3 is a perspective view of the patient monitor of FIG. 1 displaying another embodiment of a screen after activation of the symmetry control feature;

FIG. 4 is a block diagram of an embodiment of the patient monitor of FIG. 1;

FIG. 5 is a perspective view of another embodiment of a patient monitor that may employ a symmetry control feature; and

FIG. 6 is a perspective view of the patient monitor of FIG. 5 after activation of the symmetry control feature.

### DETAILED DESCRIPTION

One or more specific embodiments of the present disclosure will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

The present disclosure relates to customization of user interfaces for medical devices, such as patient monitors. According to certain embodiments, the patient monitors may include a symmetry control feature that adjusts the position of touch sensitive inputs shown on a display of the patient monitor. The symmetry control feature may allow a user to move the touch sensitive inputs from one side of the display to another to allow a user to access the touch sensitive inputs without blocking a portion of the display that displays patient physiological data. In certain embodiments, the symmetry control feature may be designed to accommodate left-handed and right-handed users.

The patient monitors may include a graphical user interface with a graphical element that may be selected to activate the symmetry control feature and move the touch sensitive inputs to an opposite side of the display. Through the graphical user interface, a user also may enter user preferences that specify the location of the touch sensitive inputs for that user. For example, a right-handed user may enter preferences specifying that the touch sensitive inputs should be displayed on the right-hand side (from a user's perspective) of the patient monitor. In another example, a left-handed user may enter preferences specifying that the touch sensitive inputs should be displayed on the left-hand side (from a user's perspective) of the patient monitor. In certain embodiments, the patient monitors may include one or more communication interfaces for receiving user identification information. Upon receiving user identification information, the symmetry control feature may retrieve user preferences associated with the user identification information and may display the touch sensitive inputs in the locations specified by the user preferences.

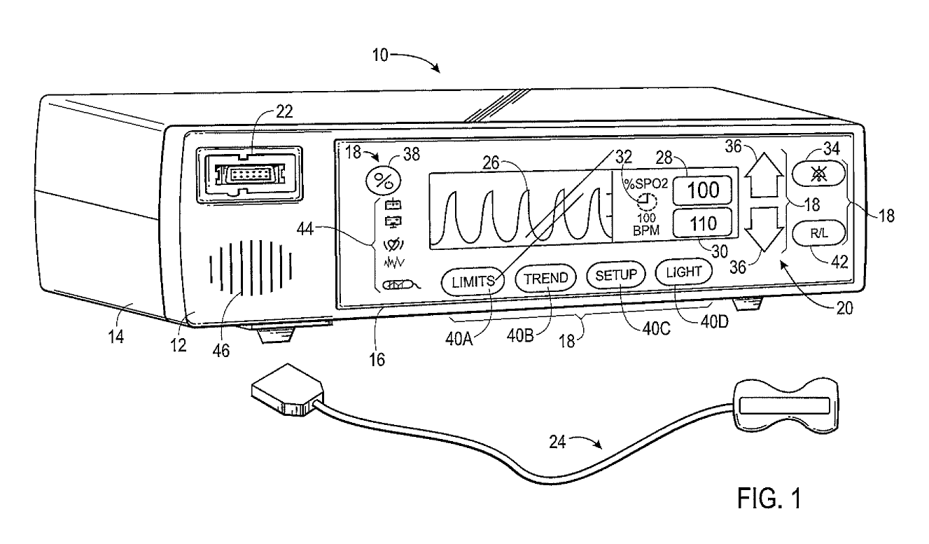

FIG. 1 is a perspective view of an embodiment of a patient monitor 10. For example, the patient monitor 10 may be a pulse oximeter, such as those available from Nellcor Puritan Bennett LLC of Boulder, Colo. As shown, the patient monitor 10 is a pulse oximeter designed to detect and monitor blood oxygen saturation levels, pulse rate, and so forth. However, in other embodiments, the symmetry control feature may be employed in other types of patient monitors, such as vital signs monitors, critical care monitors, obstetrical care monitors, or blood pressure monitors, among others. Further, the patient monitor 10 may be part of a therapeutic medical device, such as a mechanical ventilator, or anesthesia machine, among others.

The patient monitor 10 may includes a front panel 12 coupled to a body 14 of the patient monitor 10. The front panel 12 may include a display 16 that operates in conjunction with a touch screen. In certain embodiments, the display 16 may include a cathode ray tube or liquid crystal display that has a touch screen positioned in front or behind the display. Further, in certain embodiments, the touch screen may be integrated with the display 16.

The display 16 may display touch sensitive inputs 18 that may be selected by a caretaker to operate the patient monitor 10. For example, the touch sensitive inputs 18 may include graphical elements that may be pressed to change information shown on one or more screens 20 of a graphical user interface. For example, screen 20 may include a patient monitoring screen that shows processed physiological data and/or other data received through a medical device interface 22, from a patient sensor 24, or other suitable medical device, such as a therapy device. As shown, the medical device interface 22 includes a cable connection port. However, in other embodiments, the medical device interface 22 may be any suitable type of interface for connecting to a medical device. For example, in certain embodiments, the medical device interface 22 may include a wireless interface.

According to certain embodiments, the display 16 may be used to display a plethysmographic (“pleth”) waveform 26, an oxygen saturation 28, and/or a pulse rate 30. The oxygen saturation 28 may be a functional arterial hemoglobin oxygen saturation measurement displayed as units of percentage SpO₂. The pulse rate 30 may indicate a patient's pulse rate in beats per minute. The display 16 also may be used to show topic-specific screens related to the physiological data, such as a “blip” display that includes pulse amplitude blips, a real-time trend display, and an alarm limit and monitoring mode display. Moreover, the display 16 may be used to display user interface options, such as a setup and/or a configuration screen for adjusting parameters such as alarm volume, display scales, and touch sensitive input locations, among others.

In addition to displaying physiological information, the patient monitor 10 may also display information related to alarms and monitor settings on the display 16. For example, in some embodiments, the patient monitor 10 may employ SatSeconds™ by Nellcor™ to detect alarms and manage nuisance alarms. SatSeconds™ may include activation of an alarm based on limits that may include the integral of time and depth of a desaturation event and may include an indicator 32 that may serve to inform the caretaker that an SpO₂ reading has been detected outside of the limit settings. The display 16 may also include an alarm status indicator (not shown), such as a bell that flashes when an alarm condition is present. One of the touch sensitive inputs 18, such as an alarm silence graphical element 34, may be selected to silence the alarm and display an alarm silence indicator, such as a slash through an alarm symbol, on the alarm silence graphical element 34. The alarm silence graphical element 34 may then be selected again to un-silence the alarm and remove the alarm silence indicator from the graphical element 34.

In general, the touch sensitive inputs 18 may be used to control operational functions of the patient monitor 10. The touch sensitive inputs 18 may include graphical elements, such as the alarm silence graphical element 34, arrows 36, and a power key 38. For example, the arrows 36 may be selected to adjust alarm limits and/or to vary the physiological information shown on the display 16. In another example, the power key 38 may be selected to turn the monitor 10 on and off.

The touch sensitive inputs 18 also may include graphical elements 40 that may be selected to navigate through menus of the monitor 10. For example, each of the four graphical elements 40A, 40B, 40C, and 40D may be selected to display corresponding menus governing operation of the monitor 10. For example, the graphical element 40A may be pressed to display “LIMITS” information, while the graphical element 40B may be pressed to display “TREND” information. In certain embodiments, the graphical elements 40 may be selected to display operating information such as alarm limits, historic trends, setup menus, and alarm volume settings, among others. Moreover, a caretaker may select the graphical elements 40 to display various operating menus, and then may select the arrows 36 to adjust operating parameters.

The touch sensitive inputs 18 also may include a symmetry control graphical element 42 for varying the position of the touch sensitive inputs 18 on the display 16. For example, a left-handed user may prefer that the arrows 36 and the alarm silence graphical element 34 be included on the left-hand side of the display 16 to reduce blockage of the display 16 during selection of the touch sensitive inputs 34 and 36. A user may select the symmetry control graphical element 42 to move some, or all, of the touch sensitive inputs 18 to an opposite side of the display 16. Further, in certain embodiments, the locations designated for the touch sensitive inputs and the touch sensitive inputs affected by selection of the symmetry control graphical element 42 may be customized through menus of the patient monitor 10.

In addition to the touch sensitive inputs 18, the display 16 may include various status indicators 44 (e.g., display screen graphics) that facilitate operation of the monitor 10. For example, the status indicators 44 may include an A/C power indicator, a low battery indicator, an alarm silence indicator, a mode indicator, and so forth. The front panel 12 also includes a speaker 46 for emitting audible indications (e.g., alarms). In certain embodiments, the speaker 46 and/or the status indicators 44 may be located at other locations of the patient monitor 10 or on an external device.

Multiple caretakers may be responsible for a patient's care, and accordingly, several different caretakers may operate the patient monitor 10. For example, nurses may rotate based on hospital shifts. Further, the patient monitor 10 may be employed in different environments, such as a patient's hospital room, a patient's home, or an operating room. Depending on the environment, various locations of the touch sensitive inputs 18 may be desired. For example, in an operating room, it may be desirable to include touch sensitive inputs 18 on a side of the monitor that is easy to access. In another example, a left-handed user may prefer that the arrows 36 and the alarm silence button 34 be included on the left-hand side of the display 16 to reduce blockage of information, such as the physiological data 26, 28, 30, and 32 and/or the indicators 44, shown on the display 16 during selection of the touch sensitive inputs 34 and 36. Accordingly, the symmetry control graphical element 42 may be selected to vary the location of the touch sensitive inputs 18.

For example, as shown in FIG. 1, the screen 20 is setup for operation by a right-handed user, with the touch sensitive inputs 34, 36, and 42 located on the right-hand side of the display 16 to allow a user to select the touch sensitive inputs 34, 36, and 42 without reaching across the display 16. As shown in FIG. 1, the status indicators 44 are located on the left-hand side of the display 16. In response to selection of the symmetry control graphical element 42, the patient monitor 10 may display some, or all, of the touch sensitive inputs 18 on an opposite side of the display 16.

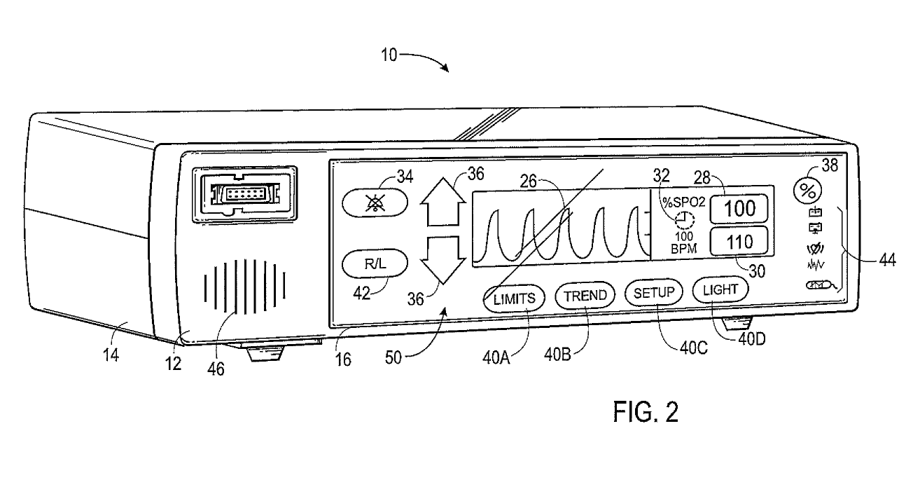

FIG. 2 depicts an embodiment of the patient monitor 10 after selection of the symmetry control graphical element 42. In response to selection of the symmetry control graphical element 42, the patient monitor 10 may display a new screen 50 that interchanges the positions of the touch sensitive inputs 18 and the indicators 44. Specifically, as shown on the screen 50, the touch sensitive inputs 34, 36, and 42, are located on the left-hand side of the display 16 while the indicators 44 are located on the right-hand side of the display 16. In certain embodiments, the screen 50 may be designed to facilitate use by a left-handed user by allowing a user to select the touch sensitive inputs 34, 36, and 42 without reaching across the display 16.

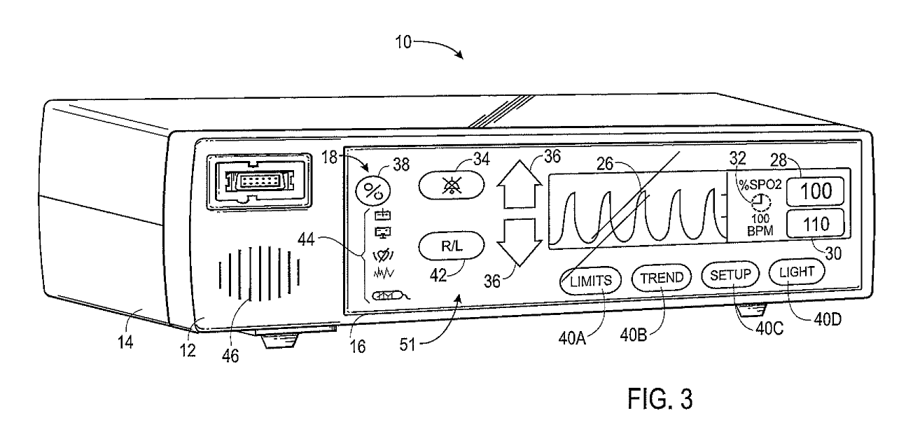

FIG. 3 depicts another screen 51 that may be displayed on the patient monitor 10 in response to selection of the symmetry control graphical element 42. From the screen 20, shown in FIG. 1, a user may select the symmetry control graphical element 42 to display the screen 51 that interchanges the positions of the physiological data 26, 28, 30, and 32 and the touch sensitive inputs 18. As shown on the screen 51, the indicators 44 may remain in the same position, while the touch sensitive inputs 18 are moved to the left-hand side of the display 16 to a position in between the indicators 44 and the physiological data 26, 28, 30, and 32. The menu graphical elements 40 have also been moved, along with the physiological data 26, 28, 30, and 32, to the right-hand side of the display 16.

In other embodiments, the relative locations of the touch sensitive inputs 18 and/or the number of touch sensitive inputs 18 moved to an opposite side of display 16 in response to selection of the symmetry control graphical element 42 may vary. For example, in certain embodiments, only the arrows 36 may move to another location, in another example, the menu graphical elements 40 may remain in a constant location. Further, other items shown on the display 16, such as the indicators 44, may be moved in response to selection of the symmetry control graphical element 42.

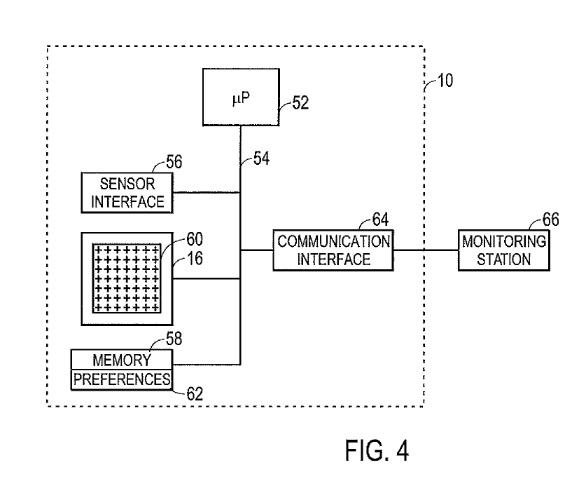

FIG. 4 is a block diagram of the embodiment of the patient monitor 10 shown in FIGS. 1-3. The patient monitor 10 generally includes a microprocessor 52 connected to an internal bus 54. A sensor interface 56 may be connected to the bus 54 and may allow the patient monitor 10 to communicate with and receive physiological data from the sensor 24 (FIG. 1). In certain embodiments, the sensor interface 56 may include components, such as a decoder for decoding signals from the sensor, algorithms or lookup tables for identifying physiological parameters, drive circuits, and signal-processing equipment, such as filters, analog to digital converters, amplifiers, queued serial modules, and time processing units, among others.

In general, the sensor interface 56 may be designed to receive input from the sensor 24 and transmit signals to the microprocessor 52 in a form that the microprocessor 52 may use to calculate and/or to determine physiological parameters, for example, based on algorithms or look-up tables stored in a memory 58. In certain embodiments, the microprocessor 52 may use the information from the sensor interface 56 to determine physiological parameters, such as SpO₂, pulse rate, respiratory effect, and so forth. The physiological parameters may then be displayed on the display 16. For example, as shown in FIG. 1, the physiological parameters, such as the pleth waveform 26, the oxygen saturation 28, and the pulse rate 30, may be shown on the display 16. The microprocessor 52 also may execute code associated with the symmetry control feature to vary the locations of the touch sensitive inputs 18. In certain embodiments, code, software, algorithms, or the like, for the symmetry control feature may be stored within the memory 58.

The memory 58 may include volatile memory, such as random access memory (RAM) and/or non-volatile memory, such as read-only memory (ROM), and the like. The memory 58 also may store components, such as layers, windows, screens, templates, elements, or other components that may be shown on the display 16. In certain embodiments, the components may be part of a graphical user interface (“GUI”) that enables a caretaker to operate the monitor 10 through a touch screen 60. For example, the GUI may include the touch sensitive inputs 18 (FIG. 1) that are shown on the display 16 and that are selectable through the touch screen 60. The touch screen 60 may receive input from a caretaker's or object's touch and may send the information to the microprocessor 52, which may interpret the touch event and perform a corresponding action. The touch screen 60 may employ any suitable type of touch screen technology, such as resistive, capacitive, infrared, surface acoustic wave, electromagnetic, or near field imaging, among others.

In certain embodiments, the memory 58 may store data representing user preferences 62. For example, the user preferences 62 may include preferred display locations for the touch sensitive inputs 18 (FIG. 1). The user preferences 62 may be entered by a user through the patient monitor 10, for example, by navigating through menus using the touch sensitive inputs 18. Upon entry, a user may save the user preferences 62 to the memory 58. Further, in certain embodiments, the symmetry control feature may store previously selected locations for the touch sensitive inputs 18 as user preferences 62.

In addition to specifying the locations for the touch sensitive inputs 18, the user preferences 62 may specify which touch sensitive inputs 18 are moved to the other side of the display 16 in response to selection of the symmetry control graphical element 42 (FIG. 1). Further, the user preferences 62 may specify the location of the touch sensitive inputs 18 based on another type of input in addition to, or instead of, selection of the symmetry control graphical element 42. For example, the user preferences 62 may store touch sensitive input locations associated with user identification information that may be received through the GUI and/or from an external device connected to the patient monitor 10 through a communication interface 64. In certain embodiments, a user may change the display language to a language written from right-to-left, such as an Arabic language, and, in response to receiving this selection, the symmetry control feature may move one or all of the touch sensitive inputs 18 to the left-hand side of the display. In another example, the patient monitor 10 may receive login information for a caretaker, and, in response to receiving the login information, the symmetry control feature may move the touch sensitive inputs 18 to locations stored as user preferences 62 corresponding to the received login information. Further, in certain embodiments, the user preferences 62 may store sizes, shapes, and/or layouts for the touch sensitive inputs 18.

The user preferences 62 also may include preferred display formats for the patient physiological data, such as display views (e.g., whether a pleth waveform or a blip bar is shown), default trend displays (e.g., whether the trend display defaults to a oxygen saturation display, a pulse rate display, a dual display, or a histogram), the display scale (e.g., the time range or amplitude range for the pleth waveform), and the display locations (e.g., where the physiological data is located on the display 16). The user preferences 62 also may include preferred operating parameters, such the pulse beep volume, the alarm volume and/or duration, the alarm limits, and the response mode, among others. Further, the user preferences 62 may include preferred user interface display formats, such as the preferred display language, icon size or symbol set, and the size of display areas and/or the touch sensitive inputs 18, among others.

The patient monitor 10 also may include the communication interface 64 that enables communication with external devices, such as a monitoring station 66. For example, the communication interface 64 may include network connections enabling wired or wireless network communications. According to certain embodiments, the monitoring station 66 may display physiological data from one or more connected patient monitors 10. The monitoring station 66 may allow a caretaker to monitor the physiological data from several patients in a single location.

In certain embodiments, login information from the monitoring station 66 may be transmitted to the monitor 10 through the communication interface 64. The microprocessor 52 may use the login information to retrieve and apply user preferences 62 associated with the login information. For example, each caretaker may have associated user preferences 62 determining the location of the touch sensitive inputs 18 on the display 16. In certain embodiments, the symmetry control feature may adjust the location of the touch sensitive inputs 18 in response to receiving the login information. However, in other embodiments, the patient monitor 10 may wait to apply the touch sensitive input locations associated with the user preferences 62 until selection of a touch sensitive input 18, such as the symmetry control graphical element 42.

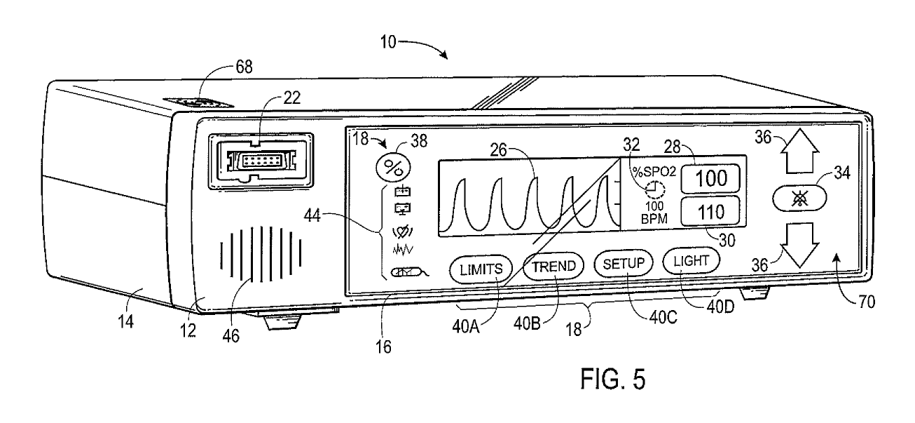

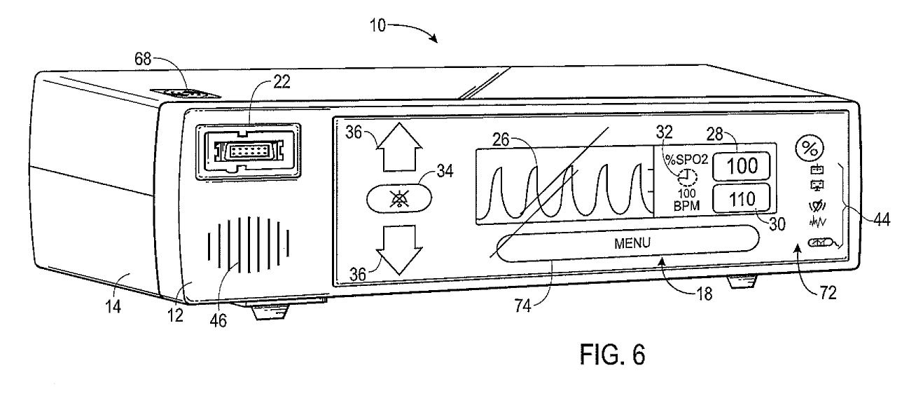

The communication interface 64 also may include other types of communication interfaces allowing communication with external devices. For example, in certain embodiments, the communication interface 64 may include a radio frequency identification (RFID) reader that reads information from RFID tags. Through the communication interface 64, the patient monitor 10 may read RFID tags to retrieve user identification information associated with individual caregivers. The symmetry control feature may then use the user identification information to retrieve touch sensitive input locations stored as user preferences 62. As described below with respect to FIGS. 5 and 6, the patient monitor 10 may then display the touch sensitive inputs 18 in the locations specified by the user preferences 62.

FIG. 5 depicts an embodiment of a patient monitor 10 with an RFID reader 68. As shown, the RFID reader 68 is located within the body 14. However, in other embodiments, the RFID reader 68 may be located within the front panel 12. As described above with respect to FIG. 4, the RFID reader 68 may be used to receive user identification information associated with user preferences 62. In response to receiving user identification information, the patient monitor 10 may retrieve the corresponding user preferences 62 and may vary the locations of the touch sensitive inputs 18 to correspond to the locations stored within the user preferences 62.

As shown in FIG. 5, the patient monitor 10 displays a screen 70 with the touch sensitive inputs 34 and 36 shown on the right-hand side of the display 16. The screen 70 may be generally similar to the screen 20 shown in FIG. 1; however, the symmetry control graphical element 42 may be omitted. In response to receiving user identification information through RFID reader 68, the patient monitor 10 may retrieve the user preferences 62 (FIG. 4) associated with the user identification information and determine a location for one or more of the touch sensitive inputs 18. The patient monitor 10 may then display a new screen 72, as shown in FIG. 6, that displays the touch sensitive inputs 18 in the locations associated with the user identification information. For example, the screen 72 now shows the touch sensitive inputs 34 and 36 on the left-hand side of the screen.

In certain embodiments, the user preferences 62 (FIG. 4) also may specify different shapes, sizes, and/or layouts for some or all of the touch sensitive inputs 18. For example, as seen by comparing FIGS. 5 and 6, the menu graphical elements 40A, 40B, 40C, and 40D have been replaced by one large menu graphical element 74. In certain embodiments, the larger size may facilitate selection of the graphical element 74 by a user wearing gloves. Moreover, in other embodiments, the patient monitor 10 may vary other settings, such as the display formats described above with respect to FIG. 4, in addition to varying the location of the touch sensitive inputs 18.

As may be appreciated, the symmetry control features described above with respect to FIGS. 1-6 may be employed within various types of patient monitors employing touch screens. As noted above, the symmetry control features may be employed to vary the display location of one or more touch sensitive inputs 18. Further, the symmetry control feature may vary the display location of other elements shown on the display 16, such as the status indicators 44 and the physiological data 26, 28, 30, and 32, to accommodate the adjusted locations for the touch sensitive inputs 18. Moreover, the relative sizes, shapes, numbers, and geometries of the GUI features, such as the status indicators, the touch sensitive inputs 18, the screens, the displays, and the windows, may vary.

What is claimed is:

1. A patient monitor, comprising:
a device body comprising one or more medical device interfaces suitable for connection to a sensor and/or a therapy device;
a touch screen capable of displaying:
patient physiological data based on input received from the sensor or the therapy device; and
one or more touch sensitive inputs for controlling operation of the patient monitor; and
a symmetry control feature that moves at least one of the one or more touch sensitive inputs to an opposite side of the touch screen in response to activation.

2. The patient monitor of claim 1, wherein the symmetry control feature is activated in response to user selection of a symmetry control graphical element.

3. The patient monitor of claim 1, wherein the symmetry control feature is activated in response to a user login.

4. The patient monitor of claim 1, comprising a radio frequency identification tag reader, and wherein the symmetry control feature is activated in response to user identification information received through the radio frequency identification tag reader.

5. The patient monitor of claim 1, comprising a network connection, and wherein the symmetry control feature is activated in response to user identification information received through the network connection.

6. The patient monitor of claim 1, wherein the symmetry control feature moves the at least one of the one or more touch sensitive inputs from one side of the patient physiological data to an opposite side of the patient physiological data in response to activation.

7. The patient monitor of claim 1, wherein the touch screen is capable of displaying indicators describing a status of the patient monitor, and wherein the symmetry control feature interchanges display positions of the indicators and the at least one of the one or more touch sensitive inputs in response to activation.

8. The patient monitor of claim 1, wherein the symmetry control feature interchanges display positions of the patient physiological data and the at least one of the one or more touch sensitive inputs in response to activation.

9. A patient monitor, comprising:
a device body comprising one or more medical device interfaces suitable for connection to a sensor or a therapy device;
a communication interface capable of receiving a user identifier;
a touch screen capable of displaying:
patient physiological data based on input received from the sensor or the therapy device; and
one or more touch sensitive inputs for controlling operation of the patient monitor; and
a symmetry control feature that determines a location of the one or more touch sensitive inputs based on the user identifier.

10. The patient monitor of claim 9, wherein the communication interface comprises a radio frequency identification tag reader.

11. The patient monitor of claim 9, wherein the communication interface comprises a wired or wireless network communication interface.

13. The patient monitor of claim 9, wherein the symmetry control feature determines the location based on a user preference that correlates the user identifier to the location.

14. The patient monitor of claim 9, wherein the symmetry control feature determines the location based on a previous location selected for the user identifier.

15. A method, comprising:
receiving patient physiological data;
displaying, on a touch screen, visual data corresponding to the patient physiological data;
receiving an input associated with a user;
selecting, based on the input, a first side of touch screen or a second side of the touch screen for one or more touch sensitive inputs controlling the display of the visual data, wherein the first and second sides are opposite from one another; and
displaying the one or more touch sensitive inputs on the selected side of the touch screen.

16. The method of claim 15, wherein receiving an input comprises reading a radio frequency identification tag.

17. The method of claim 15, wherein receiving an input comprises receiving user login information.

18. The method of claim 15, wherein receiving an input comprises receiving the input from a central monitoring station.

19. The method of claim 15, wherein receiving an input comprises receiving a touch event selecting a symmetry control graphical element displayed on the touch screen.

20. The method of claim 14, wherein selecting a first side of the touch screen or a second side of the touch screen comprises retrieving a user preference corresponding to the input.
